(19) United States
(12) Patent Application Publication
(10) Pub. No.: US 2010/0234749 A1
(43) Pub. Date: Sep. 16, 2010

(54) SYSTEM AND METHOD FOR COMBINING SERIAL ECG ANALYSIS AND ECG ORDERING

(75) Inventors: Richard Gregg, Westford, MA (US); Sophia Zhou, Camarillo, CA (US)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., Eindhoven (NL)

(21) Appl. No.: 12/738,419

(22) PCT Filed: Oct. 20, 2008
(86) PCT No.: PCT/IB08/54304

§ 371 Date: Apr. 16, 2010

Related U.S. Application Data
(60) Provisional application No. 60/982,264, filed on Oct. 24, 2007.

(30) Foreign Application Priority Data
Oct. 20, 2008 (IB) PCT/IB08/54304

(51) Int. Cl. A61B 5/0402

(52) U.S. Cl. 600/509

(57) ABSTRACT

A system and method for receiving a request for a test to be performed on a patient, retrieving information about the patient, receiving further information about the test after the test is performed and comparing the information to the further information. In addition, a system having an input device for receiving a request for a test to be performed on a patient and a management system configured to retrieve information corresponding to the patient, combine the received request and the retrieved information into a modified request, and forward the modified request to a device performing the test.

### FIELD OF THE INVENTION

The present invention relates generally to a system and method for improving the process of ordering and evaluating serial electrocardiograms (“ECG”).

### BACKGROUND

ECGs are commonly used by medical professionals to monitor the cardiac health of patients. They are the most efficient and effective tool for diagnosing cardiac arrhythmia, myocardial ischemia/infarct (heart attack), and are also useful for diagnosing a variety of other conditions. In addition to pathological changes, ECG waveforms also often present biological variations. Using a single ECG alone, the diagnostic value is limited. Examining ECG serial changes over time can help physicians make more accurate diagnosis. Comparing a current ECG to previous ECGs is a process known as serial ECG comparison.

### SUMMARY OF THE INVENTION

A method for receiving a request for a test to be performed on a patient, retrieving information about the patient, receiving further information about the test after the test is performed and comparing the information to the further information.

In addition, a system having an input device for receiving a request for a test to be performed on a patient and a management system configured to retrieve information corresponding to the patient, combine the received request and the retrieved information into a modified request, and forward the modified request to a device performing the test.

### DESCRIPTION OF THE DRAWINGS

FIG. 1 shows an exemplary embodiment of a method for requesting and evaluating serial ECGs according to the present invention.

FIG. 2 shows an exemplary embodiment of a system for receiving and processing requests for serial ECGs according to the present invention.

### DETAILED DESCRIPTION

The exemplary embodiments of the present invention may be further understood with reference to the following description and the appended drawings, wherein like elements are referred to with the same reference numerals. The exemplary embodiments of the present invention describe a system and method for improving the accuracy and efficiency of ECG analysis by automatically providing more information to ECG technicians and ECG overreaders than is typically provided. According to the exemplary embodiments of the present invention, previous ECGs are automatically retrieved for comparison, along with additional patient information. The exemplary system and method will be described in detail below.

The exemplary embodiments of the present invention may be implemented using any system having an ECG storage capability and/or a device for ECG acquisition. Exemplary systems having ECG storage capabilities may include, for example, an ECG management system, a hospital information system (HIS), an electronic medical records system (EMR), or other clinical information systems (CIS) used in physician's clinics, outpatient clinics or hospitals. Thus, the ECG management system referred to herein may be any of these types of exemplary systems or their equivalents. The ECG acquisition device may be, for example, a cardiograph, a defibrillator with diagnostic ECG capability, a patient home monitor, a patient bedside monitor, or any device with diagnostic ECG capability.

ECGs are used by medical professionals to diagnose a wide variety of conditions. In addition to being the best method for diagnosing cardiac arrhythmia, ECGs can be used to diagnose myocardial infarctions, electrolyte disturbances such as hyperkalemia and hypokalemia, conduction abnormalities such as right and left bundle branch block, ischemic heart disease, pulmonary embolism, and hypothermia.

Serial ECG analysis is the evaluation and interpretation of a patient's current ECG in conjunction with one or more previously taken ECGs. Automation of this process may be valuable to a physician or caretaker in patient care. Serial ECG analysis can help overcome false negative and false positives that can result from a single ECG analysis. Such false negatives and false positives are corrected because the current ECG is compared to a previous ECG, and existing conditions are retained if the current ECG is not significantly different from the previous ECG. Further, serial ECG analysis greatly simplifies lead reversal detection, which is difficult with a single ECG. For example, if the left arm and left leg electrodes are switched, this is very difficult for even a highly skilled overreader to detect with a single ECG, and virtually impossible for an automated algorithm. In contrast, by using the current and at least one previous ECG, this process can be automated accurately. Additionally, the advantages of serial ECG analysis are not limited to physicians and highly skilled ECG overreaders; many cardiograph operators are able to interpret ECGs and can realize the same benefits.

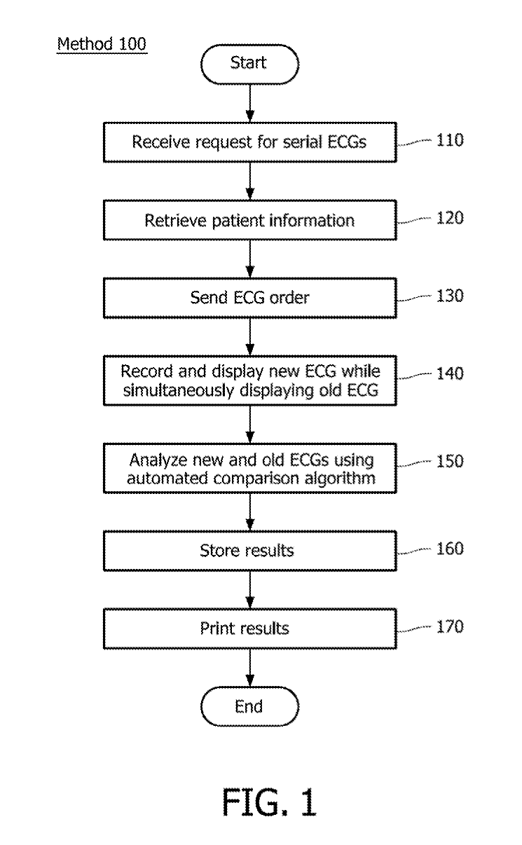

FIG. 1 shows an exemplary method 100 according to the present invention. In step 110, a request is received for periodic ECGs to be taken for a patient. Such a request is typically received from an attending physician who is treating the patient. The request may be received into an electronic system, which may be a system that is dedicated to handling ECG requests, a system that handles requests for various types of tests, or a multipurpose computer system. The input for the request may take a variety of forms, including (but not limited to) a bar code or other such identifier that may be scanned by a device which is adapted for receiving such an identifier, a patient ID number, selection of the patient's name from a list of patients in the hospital, etc. The system may store patient information beyond the identifier used to enter the request (e.g., age, gender, information about the patient's current and previous health history, etc.).

In step 120, the patient's medical information is retrieved. Information to be retrieved may include information stored about the patient in the system beyond the identifier used to select the patient, as discussed above. Most particularly, however, previous ECGs are retrieved. This step may encompass, for example, the retrieval of all previous ECGs on file for the patient, the retrieval of solely the most recent ECG, or one or more ECGs selected by the ECG management system according to a best-previous ECG algorithm. Exemplary selection criteria for the best-previous ECG algorithm may include, for example, a most recent ECG, a baseline ECG, etc.

In another exemplary embodiment, a cardiologist may have access to all the previous ECGs for the patient that are stored in a storage location in an exemplary system (described in greater detail below). Access to the previous ECGs, may also allow the cardiologist to correct one of the previously recorded ECGs which may then be marked (e.g., a flag or other electronic identifier) indicating the stored ECG is corrected. Thus, the selection algorithm may select a previously corrected ECG as the best available ECG for comparison. As will be described in greater detail below, one of the benefits of the exemplary embodiments is the elimination of false negatives and false positives based solely on the new ECG. The use of a previously corrected ECG for comparison may assist in eliminating such false negatives and positives.

In step 130, the ECG request received in step 110 is forwarded to an ECG technician. This may typically be accomplished by transmission through an ECG management system, or alternately through a general-purpose hospital computer network. The request may be automatically pushed to the ECG acquisition device, or it may be downloaded to the ECG acquisition device by the technician through a network or memory device such as a diskette, memory stick or flash card. The ECG request, as sent to an ECG technician in this step, includes the patient information retrieved in step 120.

In step 140, the ECG acquisition device operator records a new ECG. This step may typically proceed according to standard methods that are well known in the art. In step 140, the current ECG is displayed to the ECG acquisition device operator in conjunction with the one or more previous ECGs and other information retrieved in step 120. At this point, as described above, the ECG acquisition device operator may be able to determine whether any lead reversal has occurred by viewing the current ECG against the displayed previous ECG, and repeat the ECG if necessary.

In step 150, the new and previous ECGs are analyzed by an automated serial comparison algorithm. The algorithm may be designed to detect any of the conditions described above as diagnosable by ECG, or any other conditions so diagnosable. Those skilled in the art will understand that by analyzing the various features of the new ECG and/or comparing the new ECG to the old ECG, the automated algorithm may detect any number of conditions (e.g., by identifying clinically significant ECG changes). This automated step of immediate serial analysis may identify conditions that require immediate care, instead of waiting hours or even days for a cardiologist or overreader to read the new ECG.

In step 160, the new ECG and the results of the automated serial comparison are stored as part of the ECG report. In step 170, the new and previous ECGs, along with the automated serial comparison, are printed as part of the patient's records and for evaluation by the physician.

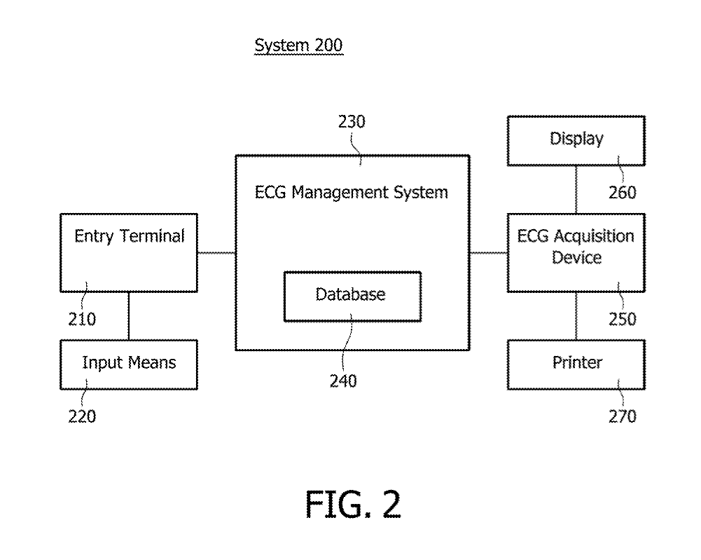

FIG. 2 shows an exemplary system 200 according to the present invention. The system 200 may operate according to the exemplary method 100 described above.

The system 200 may include an entry terminal 210. As discussed above, the entry terminal may be a computer terminal that is dedicated to receiving ECG requests, a terminal that is used for both ECG requests and requests for other types of tests, or a general-purpose computer on a hospital computer network. Actual entry of requests may be accomplished using input means 220, which may be a computer mouse and keyboard, a bar code scanner which is capable of scanning a bar code located on, e.g., a patient's chart, or any other means of data entry that are known in the art.

As described above with reference to the exemplary method 100, the request is passed from the entry terminal 210 to the ECG management system 230. The ECG management system 230 may include a database 240. The database 240 may store patient information including, but not limited to, previous ECGs for patients who are being treated, medical records pertaining to those patients, etc.

In one exemplary embodiment, the patient information stored in the database 240 may include the previous ECGs and additional information such as name, age, gender, medical history, allergies, etc., and all this information may be retrieved when needed for a new ECG. In another exemplary embodiment, the database 240 may be dedicated to storing the previous ECGs and the additional information may be stored in a different patient record system in the hospital, another central location, or locally at the ECG acquisition device 250. For example, the additional patient information may be stored on a storage device such as a bar code, a magnetic stripe, an RFID device, etc., that is included with, for example, a patient's chart, patient's wrist band, etc. The ECG acquisition device 250 may further include a data entry device (not shown) for reading the storage device. An ECG acquisition device operator may scan the storage device to retrieve the additional patient information from the storage device, while the previous ECGs may be retrieved from database 240.

When a request is received from the entry terminal 210, the ECG management system 230 retrieves information about the patient from the database 240. As described above with reference to the exemplary method 100, the information may typically include at least one previous ECG. Subsequently, the request is transmitted to a ECG acquisition device 250. This transmittal may occur automatically when the ECG management system 230 has processed a request, or when initiated by an operator of the ECG acquisition device 250. For example, when the patient arrives for administration of the ECG test, the operator may scan a bar code on the patient's chart using a bar code scanner (not shown) attached to the ECG acquisition device 250. This information may be transmitted to the ECG management system 230, which may retrieve the information corresponding to the patient ID and transmit the information to the ECG acquisition device 250.

The ECG acquisition device 250 is used to obtain an ECG for the patient; this is accomplished by following standard ECG methods known in the art. Subsequently, both current and previous ECGs may be read using the display 260. Additionally, they may be printed using the printer 270. Data, including the current ECG, may be returned to the ECG management system 230 for storage in the database 240.

It should be noted that while the exemplary system 200 shown in FIG. 2 includes a single entry terminal 210, a single ECG acquisition device 250, a single display 260, and a single printer 270, those of skill in the art will understand that a modern hospital environment may include more than one of each of the above elements. For example, as described above, the new ECG may also be stored in the database 240. Thus, an overreader may access the new and old ECG, including the specific patient information, either locally (e.g., at a terminal location in the hospital) or remotely (e.g., in any other location having access to a network including the ECG management system 230).

Through the use of the exemplary method and system, serial ECGs can be easily ordered by an attending physician. The ECG management system 230 may also be used to ensure that ordered ECG tests are performed in a timely manner when ordered by a physician (e.g., by the inclusion of a scheduling algorithm in the ECG management system 230). For example, as ECG orders are received, the ECG management system 230 may schedule the ordered ECGs based on parameters such as number of available ECG acquisition devices 250, hours of operation, etc. Further, because patient information is automatically appended by the ECG management system, an ECG technician or overreader will have more information than is typically provided with handwritten ECG orders. Finally, the ECG management-system 230 may also be used to complete the billing for ECG tests. For example, when a new ECG is stored in the database 240, the ECG management system 230 may send a message or signal to a billing system that a new ECG test has been performed on a patient, indicating that the patient or insurance company should be billed for the test.

The above exemplary embodiments have been described specifically with reference to serial ECG testing. However, it should be noted that the broader principles of the present invention are equally applicable to any type of testing which may be improved by automated ordering and/or by providing a series of results for evaluation of the development of medical conditions over time.

It will be apparent to those skilled in the art that various modifications may be made in the present invention, without departing from the spirit or scope of the invention. Thus, it is intended that the present invention cover the modifications and variations of this invention provided they come within the scope of the appended claims and their equivalents.

1. A method of performing an ECG test of a patient, comprising:
receiving a request for a test to be performed on a patient with an ECG acquisition device;
retrieving information about the patient so that it is available on the ECG acquisition device, the retrieved information including results of a previous ECG test performed on the patient;
performing a current ECG test of the patient with the ECG acquisition device;
displaying the current ECG in conjunction with the previous ECG on the ECG acquisition device; and
comparing the current ECG to the previous ECG to determine whether the new ECG is satisfactory.

2. (canceled)

3. The method of claim 1, wherein the results of a previous ECG test retrieved in the retrieving step further comprises one or more previous ECG test results.

4. (canceled)

5. The method of claim 1, wherein the comparing includes:
analyzing results of the current ECG test and results of one or more previous ECG tests using an automated serial comparison algorithm.

6. The method of claim 1, further comprising:
printing the results of the current test and the retrieved information.

7. (canceled)

8. The method of claim 1, wherein the retrieved information of the retrieving step further comprises patient data relating to one of a medical history, an age, a gender, a name, a hospital identification and an ordering physician.

9. The method of claim 1, further comprising:
storing results of the test.

10. The method of claim 5, further comprising:
providing an indication when the automated serial comparison algorithm detects a problem with the results of the test.

11. (canceled)

12. (canceled)

13. The method of claim 1, wherein the retrieving step is performed with a management system which includes a database storing the information.

14. (canceled)

15. (canceled)

16. The method of claim 1, wherein the previous ECG test of the retrieving step is the most recent previous test.

17. The method of claim 1, wherein the previous ECG test of the retrieving step is selected from a plurality of previous tests using a best-previous-test algorithm.

18. The method of claim 1, wherein the retrieved information of the retrieving step is a plurality of sets of results corresponding to a plurality of previous tests performed on the patient.

19. (canceled)

20. (canceled)
